(19) United States
(12) Patent Application Publication
(10) Pub. No.: US 2026/0235524 A1
(43) Pub. Date: Aug. 13, 2026

(54) Opioid Detection Wipes

(71) Applicant: Rockline Industries, Inc., Sheboygan, WI (US)

(72) Inventors: Douglas B. Cole, Belgium, WI (US); Jacob J. Becker, Sheboygan, WI (US)

(21) Appl. No.: 19/338,223

(22) Filed: Sep. 24, 2025

Related U.S. Application Data
(60) Provisional application No. 63/698,258, filed on Sep. 24, 2024.
(60) Provisional application No. 63/756,911, filed on Feb. 11, 2025.
(60) Provisional application No. 63/778,745, filed on Mar. 27, 2025.
(60) Provisional application No. 63/874,325, filed on Sep. 2, 2025.

(51) Int. Cl. G01N 21/78

(52) U.S. Cl. CPC G01N 21/78

(57) ABSTRACT

The present disclosure relates to drug detection devices and methods for the rapid detection of target analytes and drugs, such as opioid-containing substances, on surfaces and more specifically to drug detection devices formed with substrates including drug-detecting compositions for providing an indication of the presence of opioid-containing substances and/or other drugs contacted by the substrate. The drug-detecting compositions include one or more color-changing ingredients that produce a variable visible color change upon exposure to various target analytes, a surfactant and a polysaccharide. The color change occurs at least primarily on the substrate due to the presence in the drug-detecting composition of the surfactant and the polysaccharide that effectively binds the color-changing ingredient(s) to the substrate.

### CROSS-REFERENCE TO RELATED APPLICATIONS

The present application claims priority from U.S. Provisional Patent Application Ser. No. 63/698,258, filed Sep. 24, 2024, U.S. Provisional Patent Application Ser. No. 63/756,911 filed Feb. 11, 2025, U.S. Provisional Patent Application Ser. No. 63/778,745, filed Mar. 27, 2025 and U.S. Provisional Patent Application Ser. No. 63/874,325, filed Sep. 2, 2025, each of which is expressly incorporated herein by reference in its entirety for all purposes.

### FIELD OF THE DISCLOSURE

The present disclosure relates to detection devices and methods for the rapid detection of analytes such as opioid-containing substances on surfaces and more specifically to detection devices formed with substrates including detecting compositions for providing an indication of the presence of specific analytes including opioid-containing substances contacted by the substrate having the detecting composition thereon.

### BACKGROUND OF THE DISCLOSURE

Many pharmaceutical opioids and illegally produced opioids and mixtures of opioids and other constituents are broadly available in the public domain. Many opioids, mixtures thereof, are extremely toxic to humans and animals and can cause severe illness and fatalities form small quantities, e.g., less than 5 μg of certain type of drugs, e.g., opioids. In many cases, small quantities of opioids and mixtures thereof that are on present on a surface(s) are not readily discernable and/or visible to the naked eye. As a result, it can be difficult to avoid contact with any opioid(s) and/or other drugs present on the surface and contact with the opioid(s) and/or other drugs via direct or compromised skin contact, inhalation or ingestion by adjacent humans and animals can lead to extreme illness, unconsciousness and even death.

Drug detection tests, sometimes referred to as spot tests, have been designed to detect and differentiate between various opioids, amphetamines and other illicit drugs. These tests have been developed, modified and optimized to identify closely related classes of illicit drugs and are very useful in the prosecution of a crime. The Marquis reagent, for example, may be employed for the detection of morphine and other alkaloids, which show a red-violet color. This test has also been used as a general screening test for many drugs including various types of amphetamines. Several other tests such as Simon's reagent (for secondary amines), Mecke (for opium alkaloids), Liebermann's (for phenols), Fast blue (for cannabinoids), and Zwikker (for barbiturates) have been developed. These reagents are designed to react with functional groups of the drugs to form color compounds.

Currently, the most common drug detection tests come in the form of kits that include pouches that contain various ampules. The user must pick up and transfer the suspected drug to the pouch and break the various ampules to initiate a reaction. The solution changes color and the color itself can be used to identify the drug. These methods can be highly accurate and reproducible. However, these methods require the user to locate and pick up the drug from a surface, and this puts the user in danger of exposure that may be potentially fatal in case of highly lethal drugs like carfentanyl. This causes undue safety issues to the first responder who has to carry out the test on-site under high stress conditions. Other drug detection products use a similar technology along with an collector or sample collection device.

Current collector-based products use a cobalt thiocanate dye to indicate, by color change, contact with cocaine. Unfortunately, cobalt thiocyanate is itself a toxic substance, and these collector products cannot be used on surfaces that may come into human contact. Other collectors include other non-toxic color-changing dyes that can provide an indication of opioids on contact with the wipe on which the dye is loaded. Examples of wipes of this type are disclosed in Joy et al. US Patent Application Publication No. US2021/0302446 entitled Methods and Devices that Change Color to Indicate the Presence of Opioids and Other Narcotics, the entirety of which is expressly incorporated herein by reference for all purposes. However, these collectors including these color-changing dyes have certain limitations, including the inability of the dye to be retained on the substrate for the collector and to provide a readily discernable color change on the substrate indicating the presence of an opioid.

Other products that are available include test pens. The technology is based on antigen-enzyme binding. The small applicator tip of the pen also restricts detection of drugs to very small areas corresponding to the size of the applicator tip, negating their suitability for use on large surfaces. Also, aerosolized sprays are also available that that are also based upon antigen-enzyme binding technology. However, products that rely upon antigen-enzyme binding technology are relatively expensive.

As a result, there exists a need for a process and affordable product that allows for the rapid detection of various analytes including dangerous opioids.

### SUMMARY OF THE DISCLOSURE

According to one exemplary embodiment of the disclosure, a detection device is formed with a substrate and a drug-detecting composition loaded thereon. The drug detecting composition includes one or more color-changing ingredients capable of detecting and providing a visual indication of the presence of various drugs, such as opioids and other amine-containing drugs, on a surface that is contacted by the device. The drug-detecting composition additionally includes a binding component, such as a starch, that effectively binds the color-changing ingredient to the substrate. By effective binding the color-changing ingredient(s) to the substrate, the color change indicating the presence of the opioid occurs on the substrate where the indication is most clearly visible to the unaided eye.

According to another exemplary embodiment of the present disclosure, a method for detecting a target analyte or drug on a surface comprises contacting a surface that is suspected of containing a target analyte with a drug detection device that includes a substrate and a drug-detecting composition including one or more color-changing ingredients and a binding component that produces a visible color change on the substrate upon exposure to or contact with a drug, such as an opioid or other amine-containing drug.

According to still another exemplary embodiment of the present disclosure, a method of manufacture of a drug detection device includes forming a drug-detecting composition including one or more color-changing ingredients and a binding component. The color-changing ingredient(s) and the binding component can be formed in separate solutions that subsequently are combined to form the drug-detecting composition or can be added separately to a single solution to form the drug-detecting composition. Once formed, the drug-detecting composition can be loaded onto a suitable substrate in order effectively bind the color-changing ingredient(s) to the substrate to produce a visible color change on the substrate upon exposure to or contact with drug, such as an opioid or other amine-containing drug.

One or more exemplary embodiments of the present disclosure provide a drug detection device and associated method for the accurate detection of very small quantities of a drug, such as an opioid, that is present on a surface through a rapid color shift and/or change of a drug-detecting composition on a substrate for the device containing the color-changing composition when contacted with the drug. The drug-detecting composition remains on the substrate for the device rather than being transferred or left remaining on the surface being tested to provide a clear and readily discernable color change on the substrate if a drug is detected.

### DETAILED DESCRIPTION OF THE DISCLOSURE

The present disclosure provides an analyte and/or drug detection device, and a method for manufacture thereof for us in the rapid detection of drugs including but not limited to opioids and other amine-containing drugs on a surface. The drug detection device includes a substrate and an analyte-detecting and/or drug-detecting composition loaded onto the substrate.

### Substrate

Suitable substrates provide a solid support for the drug-detecting composition and may be selected from a wide variety of materials. The form of the substrate is not particularly limited. In one or more embodiments, the drug detection device may be formulated as a wipe, a swab, a bandage or a glove.

Substrates suitable include films, woven and nonwoven fabrics, coherent fibrous sheets, cellulosic substrates such as tissues, paper towels, coform materials, airlaid materials, bonded-carded webs, and the like. By nonwoven is meant that the layer is comprised of fibers which are not woven into a fabric but rather are formed into a membrane, sheet, substrate, mat, absorbent core or pad layer or combinations thereof. Nonexclusive examples of substrates are described in U.S. Pat. Nos. 4,775,582 and 4,853,281, 4,833,003, and 4,511,488, all of which are incorporated herein by reference. In one or more embodiments, the substrate may be in the form of a wipe, a swab, a bandge or a glove. In one or more embodiments, the substrate may also be referred to as a mat.

In one or more embodiments, the substrate is formed of fibers that may comprise natural fibers (e.g. fibers derived from plants or plant by-products, including but not limited to wood pulp, cellulose, cellulosic materials, cotton, bamboo, hemp, etc.), synthetic fibers (e.g. fibers obtained primarily from various man-made materials or from natural materials which have been further altered, including but not limited to polyolefin, polypropylene, polyethylene, polyester, polyamide, polylactic acid, lyocell, etc.) or combinations of natural and synthetic fibers. Non-limiting examples of natural materials useful in the present invention are pulp and cellulosic fibers. Non-limiting examples of cellulosic fibers include those selected from the group consisting of wood pulp fibers, bamboo fibers, cotton fibers, hemp fibers, jute fibers, flax fibers, and mixtures thereof. Non-limiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers, and mixtures thereof. Examples of some of these synthetic materials include acrylics such as acrilan, creslan, and the acrylonitrile-based fiber, orlon; cellulose ester fibers such as cellulose acetate, arnel, and acele; polyamides such as nylons; polyesters such as fortrel, kodel, and the polyethylene terephthalate fiber, dacron; polyolefins such as polypropylene, polyethylene; polyvinyl acetate fibers; polyurethane foams and mixtures thereof.

In one or more embodiments, the substrate comprises a polyester amide polymer having pendant functional groups, as described in U.S. Pat. No. 9,593,201, which is incorporated by reference herein. In one or more embodiments, the identity of the pendant groups in the polyester polymer influence the wetting nature of the substrate, which in turn influences the pick-up efficiency of the substrate. The pendant groups in the functional polyester may also influence interactions with the drug or opioid analyte, and hence the pendant groups can be used to modulate reaction times and discriminate between different classes of analytes to be detected, such as heroin and fentanyl. Advantageously, the functional polyester may be selected to favorably enhance the technology for binding of drug particles, modulation of response time, and color/tint of the substrate after contact with the drug, e.g., an opioid. By varying the pendant functional groups, the polymer fibers may be tailored for wettability, interactions with the silica particles and the response time of the color change.

In one or more exemplary embodiments, the substrate may comprise an air-laid nonwoven web. Examples include meltblown, spunbond, and bonded-carded web materials. Air-laid nonwoven sheets, and methods of making the same, are described in U.S. Pat. Nos. 3,849,241, 4,340,563, 4,443,513, 4,548,856, 4,853,281, 5,382,400, 5,575,874, 6,224,977, 6,811,638, 6,946,413, and U.S. Pat. App. Pub. Nos. 2004/0192136 A1 and 2006/0008621 A1, all of which are incorporated herein by reference.

In one or more exemplary embodiments, the substrate may comprise a coform nonwoven webs. Coform nonwoven webs may be formed by the comingling of polymeric fibers and absorbent fibers, such as polyolefin fibers and cellulosic fibers, as the fibers are entrained by a common airstream before they are deposited onto a forming surface. Coform sheet materials are described U.S. Pat. Nos. 4,100,324 and 5,350,624, and U.S. Pat. App. Pub. Nos. 2011/151596 A1, all of which are incorporated herein by reference. In one or more embodiments, the coform sheet may comprise a matrix of thermoplastic polymeric meltblown fibers and wood pulp fibers. Various suitable materials may be used to provide the polymeric meltblown fibers, such as, for example, polypropylene microfibers. In one or more embodiments, the polymeric meltblown fibers may be elastomeric fibers formed from elastomeric resins such as, for example, VISTAMAXX elastic olefin copolymer resin (available from ExxonMobil Corporation) or KRATON G styrene-ethylene/butylene-styrene and styrene-ethylene/propylene-styrene polymer resins (available from Kraton Performance Polymers, Inc.).

In one or more exemplary embodiments, the substrate may comprise hydroentangled nonwoven sheet materials. Hydroentangled nonwoven webs are sometimes referred to as spunlace fabrics. In certain aspects, hydroentangbng readily allows for the combination of different fiber types, such as combining fibers of distinct composition (e.g. polymeric fibers and wood pulp fibers) or fibers of different sizes. Hydroentangled materials, and methods of making the same, are described U.S. Pat. Nos. 3,485,706, 3,620,903, 5,009,747, 5,284,703, and 6,200,669, all of which are incorporated herein by reference.

In one or more embodiments, the substrate may comprise a water dispersible and/or biodegradable sheet material. Examples of dispersible and/or degradable nonwoven fibrous materials are described in U.S. Pat. Nos. 5,667,635, 6,750,163, and 6,960,371, and U.S. Pat. App. Pub. No. 2017/0233912, all of which are incorporated herein by reference.

In one or more embodiments, the substrate may comprise semi-synthetic or regenerated cellulose fibers, which are non-plastic and biodegradable, optionally in combination with one or more natural fibers or synthetic fibers, in an amount of between about 5% up to 100% of the fibers forming the substrate. The semi-synthetic fibers can be such as rayon, tencel, lyocell, or viscose fibers obtained by extruding or otherwise treating regenerated or modified cellulosic materials from woody or non-woody plants, as is known in the art. The absorption percentage capacity of semi-synthetic fibers, including viscose fibers, is up to 800% by weight of the dry semi-synthetic fibers.

In one or more embodiments, the substrate of the drug detection device may further include one or more optional ingredients selected from, but not limited to, color enhancers, catalysts, mechanical property modifiers, optical brighteners, anti-static agents, flame retardants, lubricants, wetting agents, softeners, mordants, and inorganic additives. The optional ingredients may be embedded within, absorbed into, dispersed upon or within, or coated upon the substrate. Chemicals and additives that are typically employed in dyeing processes and methods may be employed with the drug detection device of the present invention, including one or more of soda ash fixer, urea, Dharma dye fixative, Synthrapol, Dharma professional textile detergent, Milsoft, sodium alginate, Superclear, Calsolene oil, Bleach-Stop, Dharma color remover, optic whitener, ammonium sulfate, Jacquard silk salt, Retayne, Ludigal F, Dharma discharge paste, alum, potassium alum, and Fiber etch.

In one or more embodiments, the drug detection device includes titanium dioxide. The titanium dioxide may advantageously function as an optical brightener in the drug detection device of the present invention to enhance the visibility of any color change pf the drug-detecting composition on the substrate.

In one or more exemplary embodiments, the inorganic additive(s) includes zeolites, alumina and silica. Advantageously, in one or more exemplary embodiments, inorganic additives may be selected to increase the drug detection selectivity of the drug detection device. In one or more embodiments, the substrate may be a composite of an inorganic-polymer matrix embedded with one or more chemicals to provide indication of the presence of a drug, e.g., an opioid. In one or more embodiments, the substrate may be fabricated from polymers and silica particles.

In one or more exemplary embodiments, examples of mordants, which can optionally be used without a separate binding component, include aluminum acetate, titanium oxalate, alum, iron salts, copper salts, tin salts, siloxanes, acrylates, sodium chloride, chrome alum, tannins, vinegar, baking soda, tara powder, cream of tartar, washing soda, tannic acid, certain salts of aluminum, chromium, copper, iron, iodine, potassium, sodium, tungsten, and tin, and combinations of any of the above.

In one or more exemplary embodiments, the material forming the substrate should have sufficient mechanical strength for application to a surface without substantially degrading the form of or material forming the substrate and good processability into the desired form of the substrate. In one or more additional exemplary embodiments the material forming the substate should not be soluble in an alcoholic or an aqueous medium.

In one or more exemplary embodiments, the substrate has a polarity that is sufficient to interact with the drug-detecting composition, such that the drug-detecting composition does not leach out during processing or use of the drug detection device.

In one or more embodiments, the substrate is characterized by a dry basis weight of from about 25 g/m² to about 420 g/m², in other embodiments, from about 30 g/m² to about 300 g/m², in other embodiments, from about 35 g/m² to about 250 g/m², in other embodiments, from about 40 g/m² to about 200 g/m², in other embodiments, from about 45 g/m² to about 150 g/m², and in still further embodiments, from about 50 g/m² to about 125 g/m².

In one or more embodiments, the substrate may be a porous substrate, where porous refers to the ability of the substrate to absorb liquid. The porous structure is advantageous because: (1) it allows for efficient encapsulation of the drug-detecting composition and color-changing ingredient(s) therein that are responsible for the color change response upon contact with the drug, e.g., an opioid, (2) the retention of moisture is more efficient within the substrate, and (3) there is a resulting higher adsorption of the particles of the drug, e.g., the opioid, onto the substrate to contact the drug-detecting composition.

In one or more exemplary embodiments, the substrate may be a multi-layer substrate, where at least one layer of the substrate is porous.

In one or more additional exemplary embodiments, the substrate is at least partially non-porous and impermeable to opioids and other amine-containing drugs, as well as other drugs and harmful substances. For example, when the drug detection device is a glove, and at least partially non-porous and impermeable substrate is advantageous because it protects the user from direct contact with potentially harmful substances, such as drugs to be tested for including opioids.

If desired, the substrate may be further treated by one or more techniques as is known in the art to improve the durability, strength, hand, aesthetics, texture, and/or other properties of the substrate material. For instance, a nonwoven web may be pattern bonded or embossed by the use of heat, pressure and/or ultrasonic energy. The nonwoven substrate materials may be bonded by continuous and/or discontinuous lines, by patterns of numerous discrete elements, or other patterns as may be desired. In one or more embodiments, the nonwoven web may be bonded along the periphery of the sheet or simply across the width or cross direction of the web adjacent to the edges.

In these or other exemplary embodiments, a resin, latex or adhesive may be applied to the substrate to achieve the desired form for the substrate such as by, for example, molding, spraying or printing, to achieve the desired nature and degree of bonding. In one or more embodiments, the substrate may be treated by various other known techniques such as, for example, stretching, needling, creping, printing, dyeing, and so forth.

Certain particular, non-limiting examples of suitable generally water insoluble substrates, that can also be water-dispersible and/or biodegradable sheet materials, include nonwoven substrates, woven substrates, cloths, meshes, paper towels, napkins, cleaning pads, and the like. In an exemplary embodiment, the substrate is formed from a nonwoven material which is composed of natural fibers such pulp, cellulose, cellulosic, cotton, etc. and combined with synthetic fibers such as polypropylene, polyethylene, polyesters, polyolefins, etc. or a combination thereof. In one exemplary embodiment, the substrate material is composed of greater than 60% by weight of the substrate of natural fibers, e.g., pulp and cellulosic fibers, which can retain a sufficient amount for the drug-detecting composition for the desired detection performance of the drug detection device. Substrates made of primarily of synthetic fibers such as polyethylene terephthalate, polypropylene, polyethylene or combination thereof do not adequately encapsulate or retain the drug-detecting composition suitably to provide optimal instant color change on the substrate material. In another exemplary embodiment of the present disclosure, the fiber composition of the substrate material is formed from semi-synthetic fibers made from natural sources of regenerated cellulose with the substrate including at least 50% by weight of the substrate of semi-synthetic fibers. For example, the semi-synthetic fibers can be formed of viscose or rayon fibers, with the substrate formed of between about 60% by weight and up to 100% by weight viscose or rayon fibers. In this embodiment where the substrate is formed at primarily of viscose fibers or rayon fibers, these fibers are capable of absorbing the drug-detecting composition in amounts up to 800% by weight of the weight of the dry substrate formed at least partially of the viscose and/or rayon fibers and preferably formed of between about 60% by weight and up to 100% by weight viscose or rayon fibers.

In one or more additional exemplary embodiments, the substrate can be formed from one or more fibrous sheets that are used to form laminates with one or more additional sheet materials which can be the same or different form the material forming the fibrous sheet(s).

### Analyte-Detecting/Drug-Detecting Composition

The drug detection device of the present invention further comprises an analyte-detecting composition or, more particularly, a drug-detecting composition in which in an exemplary embodiments is formed including one or more color-changing ingredients or components and a binding component within a suitable solvent, including water and aqueous-based solvents, C₁₋₆ alcohols, glycols, and combinations thereof. In one or more exemplary embodiments, the solvent comprises one or more of water, ethanol, methanol, isopropyl alcohol, or a combination thereof. In one or more embodiments, the drug-detecting composition enables detection of the presence of one or more drugs on a surface contacted by the drug-detecting composition of the drug-detecting device, including but not limited to opioids and other amine-containing drugs. In one or more embodiments, the drug-detecting composition enables differentiation between one or more opioids and other amine-containing drugs. That is, in one or more embodiments, the drug-detecting composition enables the identification of a specific drug or compound. In one or more embodiments, the drug-detecting composition undergoes a color change in the presence of one or more opioids or other amine-containing drugs. Advantageously, the color change is rapid and easily visible to the human eye. In one or more embodiments, the color change occurs in less than 1 minute, in other embodiments, in less than 30 seconds and in other embodiments, in less than 3 seconds. In one or more embodiments, the color change is virtually instantaneous.

In one or more exemplary embodiments, the color-changing ingredient or combination of color-changing ingredients can be selected from malachite green oxalate, brilliant green, eosin yellowish, erythrosine b, methyl green, methyl violet, picric acid, crystal violet, bromocresol green, m-cresol purple, thymol blue, p-xylenol blue, cresol red, eosin bluish, quinaldine red, 2,4-dinitro phenol, 4-(dimethylamino) azobenzol, bromochlorophenol blue, bromophenol blue, congo red, methyl orange, 1-naphtholphthalein, m-cresol purple, thymol blue, p-xylenol blue, phenolphthalein, thymolphthalein, alkali blue, alizarin yellow gg, indigo carmine, epsilon blue, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, and titan yellow, among other suitable color-changing ingredients.

In one or more exemplary embodiments, the color-changing ingredient or one or a combination of color-changing ingredients in the drug-detecting composition is a curcuminoid, which is a linear diarylheptanoid, a relatively small class of plant secondary metabolites that includes curcumin, demethoxycurcumin, and bisdemethoxycurcumin, all compounds isolated from turmeric.

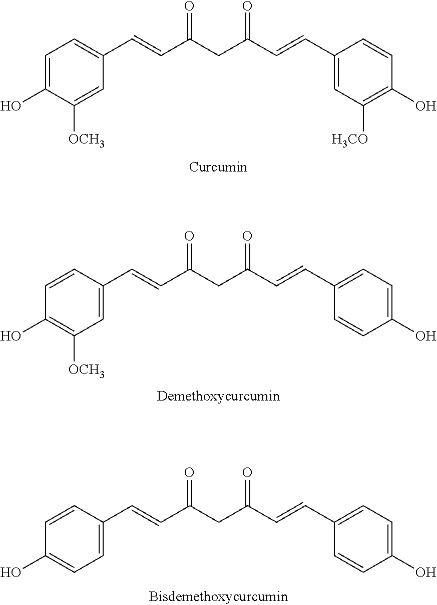 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(O)cc2)ccc1O ; O=C(/C=C/c1ccc(O)cc1)CC(=O)/C=C/c1ccc(O)cc1]

In one or more exemplary embodiments, the drug-detecting composition includes a binder component, such as a polysaccharide or cellulosic binder including but not limited to plant-based polysaccharides or starches derived from corn, potato, rice and combinations thereof, among other suitable plant-based starches. Other cellulosic binders such as carboxymethyl cellulose, hydroxyethyl cellulose, and modified polymer cellulosics and combinations thereof. Natural derived clays that contain cationic structures such as bentonite, montmorillonite, among other similar binders can also be used as the binder component. Without wishing to be bound by any particular theory, the binder component acts to or to assist in binding the drug-detecting composition to the substrate, such as by ionically and/or lipophilically binding the drug-detecting composition to pulp and cellulosic fibers and combinations thereof, such that the indication provided by the color-changing ingredient upon contact with a drug detectable by the color-changing ingredient(s) occurs at least primarily on the substrate, such that the drug-detecting composition is not deposited or left on the surface contacted by the drug-detecting device including the drug-detecting composition.

### Preparation

To form the drug-detecting composition, in one exemplary embodiment, the color-changing ingredient(s) is dissolved in a suitable solvent, including but not limited to one or more short-chain alcohols, e.g., C₁₋₆ alcohols, including ethanol, isopropanol, and combinations thereof to form a color-changing solution. The color-changing solution containing the color-changing ingredient is formed to include between about 0.4 g of the color-changing ingredient per liter of solvent (0.4 g/L) and about 2.0 g/L, with exemplary concentrations of between about 0.8 g/L and about 1.6 g/L and an exemplary concentration of 1.2 g/L (or 0.12% w/w) of color-changing ingredient in ethanol or other solvent being contemplated.

To form the binder component of the drug-detecting composition, in one exemplary embodiment the binder, e.g., a plant-based polysaccharide, is added to a suitable solvent to form a starch solution, where the solvent can be water, and more particularly deionized water. The binder is added to the water to form a binder solution of between about 12.0 g of the binder component per liter of solvent (12.0 g/L) and about 2.0 g/L, with exemplary concentrations of between about 8.0 g/L and about 4.0 g/L and an exemplary concentration of 6.0 g/L (or 0.6% w/w) being contemplated. In certain exemplary embodiments, the binder does not completely dissolve within the solvent, e.g., water, such that stirring of the binder solution forms an even dispersion or suspension of the binder within the solvent.

To form the drug-detecting composition, the color-changing solution and the binder solution are combined with one another. In one exemplary embodiment, the color-changing solution is mixed with the binder solution at ratios of between about 1:1 (color-changing solution to binder solution) and about 5:1 to form the drug-detecting solution. In another exemplary embodiment, the ratio of the color-changing solution to the binder solution in the drug-detecting composition is between about 1.5:1 to about 3:1. In still another exemplary embodiment the ratio of the color-changing solution to the binder solution in the drug-detecting composition is about 5:3.

In certain exemplary embodiments, the one or more color-changing ingredients are present in the drug-detecting composition in amounts of between about 0.05% by weight to about 5.0% by weight of the drug-detecting composition, more specifically about 0.05% by weight to about 1.0% by weight and still more specifically between about 0.1% by weight to about 0.15% by weight.

In certain exemplary embodiments, the binder is present in the drug-detecting composition in amounts of between about 0.1% by weight to about 5% by weight of the drug-detecting composition, and more specifically between about 0.2% by weight to about 0.5% by weight.

In an alternative embodiment for the formation of the drug-detecting composition, the binder can be added directly to the color-changing solution along with but separately from the amount of water required to form the binder solution to result in the desired concentration of binder in the drug-detecting composition.

In other exemplary embodiments of the drug-detecting composition, the drug-detecting composition may further include one or more optional ingredients selected from, but not limited to, color enhancers, catalysts, mechanical property modifiers, optical brighteners, anti-static agents, flame retardants, lubricants, wetting agents, softeners, mordants, and inorganic additives. In one or more embodiments, one or more optional ingredients may be selected to provide a desired pH (e.g., between pH 6 and pH 8) or ionic strength, such as a suitable base or acid, including citric acid.

According to another aspect of the present disclosure, a drug detection system is provided for rapid detection of less than high grade opioids using a substrate, such as a wet wipe, carrying a drug-detecting composition including one or more specialized color changing ingredients that provide a color change on the substrate in the presence of drugs, including opioids such as fentanyl, cocaine, methamphetamine, and other opioids and combinations thereof. As many drugs to be detected by law enforcement have significantly lower purity levels from pharmaceutical grade drugs or compounds due to other drugs or ingredients present in conjunction with the drugs to be tested for, it is necessary to provide a drug detection system that can detect these lower grade drugs even in the presence of other drugs or ingredients that might otherwise prevent an accurate detection of the drugs, e.g., the opioids. To do so, in one particular exemplary embodiment a drug-detecting composition for use on a substrate contains a unique color-changing ingredient, or combination of color-changing ingredients that changes color in the presence of an opioid or other drug to be tested for, such as curcumin, at a level between 0.05% to 5.0% by weight of the drug-detecting composition formulation, preferably 0.05% to 1.0% by weight of the drug-detecting composition and more preferably 0.1% to 0.15% by weight of the drug-detecting composition.

According to one or more exemplary embodiments of the novel drug-detecting composition disclosed herein, the binder is present in the drug-detecting composition at a level of 0.1% by weight to 5.0% by weight of the drug-detecting composition, preferably 0.2% to 0.5% by weight of the drug-detecting composition.

One or more of the exemplary embodiments of the novel drug-detecting composition disclosed herein may include one or more surfactants, such as anionic surfactants, amphoteric surfactants, zwitterionic surfactants, nonionic surfactants and/or combinations thereof. In one embodiment, when amphoteric surfactants are utilized, the pH of the drug-detecting composition is selected and/or adjusted in a suitable manner to enable the amphoteric surfactants to have anionic properties. In another embodiment the one or more surfactants are preferably anionic surfactants, optionally to the exclusion of any non-ionic surfactants or any other surfactants other than anionic surfactants, and more preferably one or more anionic surfactants with carbon chain lengths from C₈ to C₁₆, including anionic surfactants with a carbon chain length of C₁₂, and combinations thereof. The one or more surfactants can be present in the drug-detecting composition from about 0.1% to about 5.0% by weight of the drug-detecting composition, preferably about 0.5% to about 2.0%, by weight of the drug-detecting composition and more preferably 0.75% to 1.25% by weight of the drug-detecting composition. In one particular exemplary embodiment, the one or more surfactants are one or more anionic surfactants selected from linear alkylbenzene sulfonates, alpha-olefin sulfonates, alkyl sulfates, alcohol ethoxy sulfates, secondary alkanesulfonates, alpha-sulfo fatty acid methyl esters, alkyl- or alkenyl succinic acids, and mixtures thereof. In another exemplary embodiment, the one or more surfactants are one or more anionic alkyl sulfates and/or salts thereof, such as sodium lauryl sulfate, and/or anionic alkyl phosphates present in the above amount, optionally along with a nonionic surfactant selected from polyoxyethylene sorbitan fatty acid esters, such as polyoxyethylene sorbitan monooleate (Polysorbate 80), polyethylene glycol sorbitan monolaurate, polyethylene glycol sorbitan monopalmitate, polyethylene glycol sorbitan monooleate, preferably polyethylene glycol sorbitan monooleate, and mixtures thereof.

The nonionic surfactant, when present, can be present in an amount of from about 1.0% to 15.0% by weight of the drug-detecting composition, preferably about 5.0% to 10.0% by weight of the drug-detecting composition, and more preferably 5.0% to 7.0% by weight of the drug-detecting composition. The addition of the one or more anionic and/or nonionic surfactants and combinations thereof, provides lower surface tension of the disclosed solution for improved wettability of the nonwoven material forming the substrate and unexpectedly, provides additional solubility of the insoluble portion of color-changing ingredient, e.g., curcumin, that is not soluble in short chain alcohols used as the solvent, or portion of the solvent, or wetting agent of the drug-detecting composition described previously. In addition, the anionic surfactants and/or nonionic surfactants provide increased wettability of the particulates and/or surface of the color-changing ingredient, e.g., curcumin, for improved color change detection when contacted by or with the drug, e.g., the opioid, and preferably to effectively detect very low levels of drugs, e.g., fentanyl, when combined with other uncontrolled substances, drugs, ingredients and/or impurities to form a mixture including the drug/opioid being tested for. Further, when both anionic and/or amphoteric and nonionic surfactants are present, in one exemplary embodiment the ratio of nonionic surfactant to anionic surfactant present in the drug-detecting composition can be from about 25 parts nonionic surfactant to 0.5 parts anionic and/or amphoteric surfactant, and more preferably 22 parts nonionic surfactant to 1 part anionic and/or amphoteric surfactant

The novel drug-detecting composition contains other ingredients such as water and short-chain alcohols such as ethanol, isopropanol, or combinations thereof. The alcohol levels in the composition are between about 7.50% and about 65.0% by weight of the drug-detecting composition and more specifically between about 10.0% and about 40.0% by weight of the drug-detecting composition, preferably between about 15.0% and about 30.0% by weight of the drug-detecting composition, and more preferably, about 20.0% to bout 25.0% by weight of the drug-detecting composition which has been found enhance the solubilization of the one or more color-changing ingredient(s) in the drug-detecting composition. This enhanced solubilization is further enhanced when these alcohol levels are combined with the disclosed surfactant(s), e.g., the anionic surfactant, such as sodium lauryl sulfate, to solubilizes additional amounts of the color-changing ingredient(s) allowing the solubilized color-changing ingredient(s) in the drug-detecting composition to be evenly distributed on and within the substrate, optionally formed of the nonwoven material, to enhance the effectiveness of the detection capability over the entire surface area of the substrate.

In certain exemplary embodiments of the disclosure, the aforementioned drug-detecting composition can be impregnated into a substrate formed of a nonwoven material which is composed of natural fibers such pulp, cellulose, cellulosic, cotton, etc. and combined with synthetic fibers such as polypropylene, polyethylene, polyesters. Polyolefins, etc. or a combination thereof. The preferred nonwoven composition is 100% viscose fibers which delivers the best performance for this application.

In certain exemplary embodiments of the disclosure, the aforementioned drug-detecting composition is incorporated into the nonwoven substrate, such as the 100% viscose fiber substrate, at a level between 1.0 gram to 5.0 grams of solution per 1.0 gram of dry nonwoven material. Other exemplary embodiments contain preferably 1.0 gram to 4.0 grams of drug-detecting composition per 1 gram of dry nonwoven material, more preferably 1.0 gram to 3.0 grams of drug-detecting composition per 1 gram of dry nonwoven material. In still a further exemplary embodiment, the drug-detecting composition loading level on the nonwoven substrate is from 2.0 grams to 3.0 grams of the formulation per 1 gram of dry nonwoven material. For the reasons stated above these embodiments result in very low amounts of the drug-detecting composition being transferred to a surface being tested using the loaded substrate, such that the rapid color change in presence of an opioid or other drug takes place/is present on the substrate, e.g., wipe, where it can readily be seen, rather than on the surface. In one particular embodiment, the combination of curcumin (optionally combined with or substituted for by color-changing ingredients eosin Y and cresol red) and a plant-based polysaccharide is enhanced by the presence and action of the surfactant with the alcohol levels to provide chemical binding to an opioid or other drug molecule which results in a color-changing chemical reaction between the opioid and color-changing ingredient(s). The color-changing ingredients and plant-based polysaccharide combination when contacted with an opioid results in an unexpected color change or shift virtually instantaneously and also exclusively on the nonwoven surface from a yellowish color to deep red color. In addition, the substrate including the drug-detecting composition for rapid detection of opioids can be packaged in many formats such as a wet wipe in single sachets, flexible packages, and semi-ridged canisters, among others.

One exemplary embodiment of the drug-detecting composition according to the present disclosure is as follows:

| Ingredient | % wt |
|---|---|
| Ethanol | 60.000 |
| Deionized Water | 39.650 |
| Corn Starch | 0.250 |
| Curcumin | 0.100 |
| Citric Acid | Water |
| | pH to 6.0 |

Another exemplary embodiment of the drug-detecting composition according to the present disclosure is as follows:

| Ingredient | % wt |
|---|---|
| Ethanol | 23.000 |
| Deionized Water | 67.200 |
| Corn Starch | 0.250 |
| Curcumin | 0.300 |
| Citric Acid | Water pH to |
| | 6.7 ± 0.2 |
| Polysorbate 80 | 8.00 |
| Sodium Lauryl Sulfate | 1.25 |

Still other exemplary embodiments of the drug-detecting composition according to the present disclosure are as follows:

| Ingredient | % wt | Function |
|---|---|---|
| Ethanol | 20.16 | Solvent |
| Deionized Water | 77.60 | Solvent |
| Maltodextrin | 0.043 | Builder/ |
| | | Binder |
| Optional Additional Color- | 0-0.043 | Indicator |
| Changing Ingredient (e.g., | | |
| curcumin) | | |
| Cresol Red | 0.043 +/− 0.025 | Indicator |
| Eosin Y | 0.180 +/− 0.025 | Indicator |
| Sodium Lauryl Sulfate | 1.90 | Surfactant |
| Citric Acid | 0.030 | pH Adjuster |

| Ingredient | % wt | Function |
|---|---|---|
| Ethanol | 21.00 | Solvent |
| Deionized Water | 72.40 | Solvent |
| Maltodextrin | 0.043 | Builder/ |
| | | Binder |
| Optional Additional Color- | 0-0.043 | Indicator |
| Changing Ingredient (e.g., | | |
| curcumin) | | |
| Cresol Red | 0.043 +/− 0.025 | Indicator |
| Eosin Y | 0.180 +/− 0.025 | Indicator |

| Ingredient | % wt | Function |
|---|---|---|
| Sodium Lauryl Sulfate | 6.30 | Surfactant |
| Citric Acid | To adjust | pH Adjuster |
| | water pH to | |
| | 6.7 ± 0.2 | |

One or more components for the drug-detecting composition, such for example, the ethanol and sodium laurel sulfate, can be added to the composition as water/chemical blends.

### Preparation

The drug-detecting composition may be coated, impregnated or loaded onto a substrate in any of the commonly used methods for forming wet wipes substrates, such as dip and nip, spraying, ink jet printing, etc. The drug-detecting composition may also be physically adsorbed or covalently conjugated onto latex, silica, cellulosic fibers or other polymeric fibers, which may be placed on or in the substrate generally or in a pattern, whether or not employed as a wet wipe.

### Pre-Treatment

In one or more embodiments, the substrate may be pre-treated prior to applying the drug-detecting composition. Any of the optional ingredients discussed above for the substrate may be applied in a pre-treatment step.

### Dry Device Preparation

In one or more embodiments, the drug detection device is prepared by a method that includes the steps of (1) preparing a drug-detecting composition as described previously; (2) applying the drug-detecting composition to a substrate; and (3) removing some or all of the solvent. In one or more embodiments, the solvent may be removed through evaporation. In one or more embodiments, the step of removing the solvent may be accomplished with heating. In one or more embodiments, the step of removing the solvent may be accomplished in a vacuum chamber. In one or more embodiments, the step of removing the solvent may be accomplished at room temperature and pressure.

### Wet Device Preparation

In other embodiments, the drug detection device is prepared by a method that includes the steps of (1) preparing the drug-detecting composition; and (2) applying the drug-detecting composition to a substrate. However, rather than removing the solvent from the substrate to form a dry device, at least some of the liquid is left on the substrate, such that the resulting device is moist. The moist device may then be enclosed in an airtight package for storage and/or shipment to retain the moisture in the device until the package is opened before use.

In certain exemplary embodiments for the wet drug detection device, the aforementioned drug-detecting composition is incorporated into the substrate, e.g., a nonwoven material, at a level between about 1.0 gram to about 8.0 grams of drug-detecting composition per 1.0 gram of dry nonwoven material, specifically at a level between about 1.0 gram to about 7.0 grams of drug-detecting composition per 1.0 gram of dry nonwoven material, more specifically at a level between about 1.0 gram to about 6.0 grams of drug-detecting composition per 1.0 gram of dry nonwoven material, and even more particularly at a level between about 1.0 gram to about 5.0 grams of drug-detecting composition per 1.0 gram of dry nonwoven material. In one additional exemplary embodiment of the disclosure, the loading of the drug detecting liquid composition is 4.0 grams per 1.0 gram of dry, nonwoven material, i.e., a solution loading of the drug-detecting composition on the wipe of 400%, which can be formed from 100% viscose fibers.

### Post-Treatment

In one or more embodiments, the substrate may be post-treated after applying the drug-detecting composition in either the of the dry or wet preparation methods. Any of the optional ingredients discussed above for the substrate may be applied in a post-treatment step.

In one or more embodiments of either of the above dry and wet methods of preparation, a subsequent washing step may follow the step of applying the drug-detecting composition to the substrate. In one or more embodiments, the dye may be fixed onto the substrate by washing the dye/substrate with a fixative, detergent, wetting agent, surfactant, or combination thereof. Examples of fixatives include detergents such as Synthrapol SP and Retayne.

### Method of Use

The drug detection device of the present invention is useful to indicate the presence of a target analyte, e.g., an opioid or other amine-containing drug, on a surface contacted by the drug detection device. Thus, the present invention also provides a method for detecting a target analyte, such as a controlled substance, illicit drug, or other compound, on a suspected surface. The method includes the steps of contacting a surface that is suspected of containing or having a target analyte thereon with a drug detection device that includes a substrate carrying a drug-detecting composition having a color-changing ingredient that is capable of producing a visible color change to at least a portion of the substrate upon exposure to/contact with the target analyte(s). The color change occurs at least primarily on the substrate due to the presence in the drug-detecting composition of a binder component that effectively binds the color-changing component to the substrate, as described previously. Exemplary embodiments of the substance and form of the target analytes, substrates, color-changing ingredients, and color changes are as described herein.

In one or more embodiments, applying a wetting agent to either or both of the drug detection device and the suspected surface prior to contact of the drug detection device with the surface can facilitate the reaction of the target analyte and the color-changing ingredient, particularly in the case of a dry drug detection device, and thus speeds up and/or enhances the color change. In one or more embodiments, the method of the present invention includes the step of applying a wetting agent to the suspected surface prior to the step of contacting the surface with the drug detection device. In these or other embodiments, the drug detection device itself can also include a wetting agent, and is provided in a sealed, airtight package or container. The method of the present invention may further include the step of removing the drug detection device from the airtight package prior to use. In one or more embodiments, the wetting agent is non-toxic. In one or more embodiments, the wetting agent is an aqueous wetting agent. In one or more embodiments, the wetting agent comprises water, a C₁₋₆ alcohol, such as methanol, ethanol, propanol, isopropanol, or a combination thereof.

In one or more embodiments, the drug detection device may be in the nature of a dry or pre-moistened/wet wipe, sheet, mat, swab or glove. As further described hereinbelow, a user may simply bring the drug detection device into contact with a suspected drug-containing surface. In one or more embodiments, all of the surfaces of the substrate may be uniform in terms of the ability of the surface to change color. In these embodiments, any portion of the substrate may be employed in the method of use. In other embodiments, one or more portion of the surfaces of the substrate may be configured to contact the surface and optionally change color. The portion of the substrate that is configured to change color upon contact with an opioid or other amine-containing drug may be referred to as the color-changing portion. For purposes of this specification, reference to a change of color for the drug detection device should be interpreted to mean a change of color for at least the color-changing portion of the device.

If a target analyte, e.g., an opioid or other amine-containing drug, is present on the suspected surface, the detection device, or at least the color-changing portion of the device, will change color from a first color to a second color.

In one or more embodiments, the drug detection device may originally be generally yellow in color, and upon contact with a target analyte, the target analyte reacts with the color-changing ingredient(s) of the drug-detecting composition and the color of those areas of detection device containing the drug-detecting composition and contacted with/by the target analyte(s) becomes a different color, e.g., red. In one or more embodiments, contact with a target analyte results in a color change from orange to green. In one or more embodiments, contact with a target analyte results in a color change from orange to blue. In one or more embodiments, contact with a target analyte results in a color change from blood red to yellow. In one or more embodiments, contact with a target analyte results in a color change from blood red to white. In one or more embodiments, contact with a target analyte results in a color change from red to blood red. In one or more embodiments, contact with a target analyte results in a color change from white or off-white to blue.

Advantageously, the change in color may be virtually instantaneous, and does not require any additional heating, or addition of acids or bases. In one or more embodiments, the color change is readily visible on the substrate and can be discerned by the human eye.

### Target Analytes

Target analytes, i.e., the substance to be detected by the detection device, that may be detected by the detection devices and methods of the present invention include opioids and other amine-containing drugs. Many of these substances are used medically for pain relief, can be addictive, and are controlled substances. Overexposure may result in a fatal overdose. Examples include opiates, which refers to drugs that can be derived from opium. Specific examples of opioids and other amine-containing drugs include morphine, semi-synthetic and synthetic drugs such as hydrocodone, oxycodone, fentanyl, carfentanyl, acetyl fentanyl, tetrahydrofuran fentanyl, cocaine, methamphetamine and their analogues, as well as drug antagonists such as naloxone, and endogenous peptides such as the endorphins. In one or more embodiments, the target analyte includes heroin, fentanyl, oxycodone (OxyContin®), hydrocodone (Vicodin®), codeine, morphine, or a mixture or derivative thereof. In one or more embodiments, the target analyte includes one or more tertiary amine groups.

Advantageously, one or more embodiments of the present invention provide a safe and reliable way for first responders to detect the presence of various drugs, such as opioids and other amine-containing drugs using only non-toxic ingredients, thus providing a warning to avoid inadvertent exposure to potentially lethal drugs. The drug detector devices of the present invention are cost-effective and easy-to-use. In certain embodiments, where the drug detection device is a wipe or glove, pad, swab, or towel, among others large surface areas may be tested, such as table tops, cars, prison cells, etc. In other embodiments, the drug detection device is a swab that is adapted for hard to reach areas such as car consoles, pant pockets, etc. Microgram amounts of the target analyte can be effectively and accurately detected. The drug detection device may be carried safely in a patrol car, an ambulance or on a person.

In one or more additional embodiments of the disclosure, the drug detection device prepared as a wet device can be allowed to dry. After drying, the detection device, whether formed as a wet device and dried, or as a dry device, can be rewetted, e.g., with water, to moisten the substrate of the detection device and the drug-detecting composition contained thereon. The re-moistened wipe can then be applied to a surface to enable the drug-detecting composition to detect the presence of the target analyte on the surface through a color-change of the drug-detecting composition on the substrate.

In still one or more additional embodiments of the disclosure, after an initial use of the detection device on a surface, the drug detection device can be reconditioned and reused for additional drug detection usages after rinsing the used drug detection device, e.g., with water. More specifically, the drug-detecting composition on the used drug detection device changes color when contacting a target analyte, e.g., an opioid, on the surface contacted by the detection device. Upon rinsing the drug detection device with water, the target analyte is removed from the drug detection device such that the drug-detecting composition, and in particular the portion of the color-changing agent, e.g., curcumin, contacted by the target analyte, returns to its original color. The rinsed drug detection device can subsequently be re-used to perform additional detection uses, with subsequent rinsing of the drug detection device after each use.

In each embodiment, the ability of the drug-detecting composition to bind to the substrate enables the drug-detecting composition to remain on the substrate without evaporating off of the substrate when dried or being washed off the substrate when re-wetted. Separate from or in combination in whole or in part with any of the prior disclosed exemplary embodiments, according to still a further embodiment a drug-detection/wet wipe system is provided for rapid detection and general identification of drugs such as opioids using a combination of specialized color changing ingredients in a drug-detecting composition disposed on a nonwoven wipe substrate that is contacted with a surface on which can be disposed detectable amounts of certain drugs, including but not limited to opioids such as fentanyl, cocaine, methamphetamine, and other opioids and combinations thereof. The mechanism of operation of the wet wipe system for the detection and identification of the presence of the drug is a color change and/or shift of the initial color of the color-changing ingredients present on the nonwoven surface after contacting a surface suspected on having one or more drugs thereon, where the particular color change on the substrate is dependent on the structure/form of the drug, e.g., opioid contacted by the wipe. For example, each indicator is more sensitive to a particular opioid, such that the color shift in the color-changing ingredients of the drug-detecting composition varies depending on the opioid or mixtures thereof present e.g., fentanyl, fluoro-fentanyl, xylazine, meth, cocaine, can be various shades of red e.g., orange, pink, light red.

To perform in this manner, the wet wipe includes multiple unique color changing ingredients on the substrate for the wipe. For example, in one exemplary embodiment the color-changing ingredients present in the drug-detecting composition on the substrate can include, but are not limited to, a combination of curcumin, present in one exemplary embodiment at a level between 0.01% to 3% by weight of the drug-detecting composition, preferably 0.02% to 2% by weight and more preferably 0.03% to 0.1% by weight, cresol red, present in one exemplary embodiment at a level between 0.01% to 3% by weight of the drug-detecting composition, preferably 0.02% to 2% be weight and more preferably 0.03% to 0.1% by weight, and eosin y, present in one exemplary embodiment at a level between 0.05% to 5% by weight of the drug-detecting composition, preferably 0.1% to 2% be weight and more preferably 0.15% to 0.5% by weight. While these three color-changing ingredients, or combinational pairs thereof, can be used together exclusively in the drug-detecting composition present on the substrate to form the wipe, it is also contemplated that other color-changing ingredients can be included in the drug-detecting composition as well.

In any of the exemplary embodiments described above, another ingredient in the formula for the drug-detecting composition is a binder component formed from plant-based polysaccharide such as corn starch, rice starch, potato starch, or more preferably a dextrin component, such as a cyclodextrin and/or a maltodextrin. In one particular exemplary embodiment, the plant-based polysaccharide is a dextrin, such as a cyclodextrin, e.g., alpha-, beta- or gamma-, or combinations thereof, or maltodextrin present at a level of 0.01% to 2% by weight of the drug-detecting composition, and more preferably present in an amount of 0.02% to 0.1% by weight.

In any of the exemplary embodiments described above, another ingredient in the formula for the drug-detecting composition is one or more surfactants. In one particular exemplary embodiment, the one or more surfactants comprises at least one anionic surfactant present at a level of from about 1% to about 10% by weight of the drug-detecting composition, preferably about 3% to about 8% by weight, and more preferably 5% to 7% by weight. The anionic surfactant is selected from the group consisting of linear alkylbenzene sulfonate, alpha-olefin sulfonate, alkyl sulfate, alcohol ethoxy sulfate, secondary alkanesulfonates, alpha-sulfo fatty acid methyl esters, alkyl- or alkenyl succinic acid, and mixtures thereof, where in a particularly preferred embodiment the anionic surfactant is an alkyl sulfate.

The formula for the drug-detecting composition also contains other ingredients such as water and short-chain alcohols such as ethanol, isopropanol, or combinations thereof. In one particular exemplary embodiment, the alcohol is present in an amount greater than 50% by weight of the drug-detecting composition and operates to solubilize the color-changing ingredients in the drug-detecting composition and allows the solubilized color-changing ingredients in solution to be evenly distributed on and within the nonwoven material forming the substrate for the wipe. The forementioned formula for the drug-detecting composition is impregnated into a nonwoven material which is composed of natural fibers such pulp, cellulose, cellulosic, cotton, etc. and combined with synthetic fibers such as polypropylene, polyethylene, polyesters. Polyolefins, etc. or a combination thereof. The preferred nonwoven composition is 100% viscose fibers which deliver the best performance for detection of a drug on the surface contacted by the wet wipe and drug-detecting composition contained thereon. The forementioned formula for the drug-detecting composition is incorporated into the preferred nonwoven at a level between 1 gram to 5 grams of the drug-detecting composition per 1 gram of dry nonwoven material, preferably 1 gram to 4 grams of drug-detecting composition per 1 gram of dry nonwoven material, more preferably 1 gram to 3 grams of drug-detecting composition per 1 gram of dry nonwoven material. The most preferred solution loading level for the drug-detecting composition onto the nonwoven substrate is 2 grams to 3 grams of drug-detecting composition per 1 gram of dry nonwoven material. The preferred embodiment results in very low amounts of the drug-detecting composition being transferred to a surface from the wet wipe, such that the rapid color change of the drug-detecting composition in presence of an opioid takes place on the wipe and not on the surface being tested. The combination of curcumin, cresol red, eosin Y, and plant-based polysaccharide, e.g., dextrin, provides chemical binding to an opioid molecule which results in a color-changing chemical reaction between the opioid and curcumin, cresol red, and eosin Y. The color changing or shift between the curcumin, cresol red, eosin Y, and plant-based polysaccharide combination in contact with an opioid, results in an unexpected color change or shift on the nonwoven surface which color is dependent on the structure of the opioid contacted. The unexpected color change or shift on the nonwoven surface can vary in an exemplary embodiment of the drug-detecting composition from a starting color of yellow, or yellow-orangish color to an indication color of ruby red, dark red, magenta, scarlet, rust, red-orange, in which the particular shade of the indication color change will be indicative of the opioid or other drug detected, such as that illustrated in Table 1, below.

TABLE 1
Chart of Wipe Starting Color (Pre-Test) and Visible Color
Change on Indicated On Wipe With Associated Drug Compounds

| | | | |
|---|---|---|---|
| Starting | Compound | Indication | Color |
| Color | Tested | Color Change | Description |

| | |
|---|---|
| Fentanyl | Red |
| Methamphetamine | Red-Orange |
| Cocaine | Purple |
| Lidocaine | Plum |

The wet wipe for rapid detection of opioids of the present disclosure can be packaged in many formats such as single sachets, flexible packages, semi-ridged canisters, etc., and on which the color chart in Table 1 can be disposed as a reference for an individual utilizing the wipe on a surface suspected of having a drug on the surface thereof.

Various modifications and alterations that do not depart from the scope and spirit of this invention will become apparent to those skilled in the art. This invention is not to be duly limited to the illustrative embodiments set forth herein.

What is claimed is:

1. A drug detection device for detecting a target analyte on a surface, the device comprising:
a) a substrate; and
b) a drug-detecting composition disposed on the substrate, the drug-detecting composition comprising:
i. one or more color-changing ingredients that produce variable visible color changes upon exposure to various target analytes;
ii. a binder component; and
iii. at least one surfactant.

2. The drug detection device of claim 1, wherein the is a plant-based polysaccharide.

3. The drug detection device of claim 2, wherein the plan-based polysaccharide is a dextrin.

4. The drug detection device of claim 3, wherein the dextrin is present in an amount of between about 0.01% w/w to about 2.0% w/w of the drug-detecting composition.

5. The drug detection device of claim 1, wherein substrate is formed of at least 50% w/w viscose fibers.

6. The drug detection device of claim 5, wherein the substrate is formed of from about 60% w/w up to 100% w/w viscose fibers.

7. The drug detection device of claim 5, wherein the substrate is in the form of a pre-moistened wipe, a swab, or a glove.

8. The drug detection device of claim 1, wherein the one or more multiple color-changing ingredients comprises cresol red and eosin Y.

9. The drug detection device of claim 8, wherein the cresol red is present in an amount of between about 0.01% w/w to about 3% w/w of the drug-detecting composition.

10. The drug detection device of claim 8, wherein the eosin Y is present in an amount of between about 0.05% w/w to about 5% w/w of the drug-detecting composition.

11. The drug detection device of claim 1, wherein the at least one surfactant is an anionic surfactant.

12. The drug detection device of claim 11, wherein the anionic surfactant has a carbon chain length of from C₈ to C₁₆.

13. The drug detection device of claim 12, wherein the anionic surfactant has a carbon chain length of C₁₂.

14. The drug detection device of claim 11, wherein the anionic surfactant is selected from the group consisting of: linear alkylbenzene sulfonates, alpha-olefin sulfonates, alkyl sulfates, alcohol ethoxy sulfates, secondary alkanesulfonates, alpha-sulfo fatty acid methyl esters, alkyl- or alkenyl succinic acids, and mixtures thereof.

15. The drug detection device of claim 1, wherein the at least one surfactant comprises:
a) an anionic surfactant; and
b) a nonionic surfactant.

16. The drug detection device of claim 15, wherein the ratio of nonionic surfactant to anionic surfactant present in the drug-detecting composition is from about 25 parts nonionic surfactant to about 0.5 parts anionic surfactant.

17. The drug detection device of claim 1, comprising:
a) ethanol;
b) deionized water;
c) maltodextrin;
d) cresol red;
e) eosin Y;
f) sodium laurel sulfate; and
g) a pH buffer.

18. A method for detecting a target analyte on a surface, the method comprising the step of: contacting a surface that is suspected of containing a target analyte with the drug detection device of claim 1.

19. The method of claim 18, further comprising the steps of:
a) rinsing the drug detection device after contacting the surface with the drug detection device to remove any target analyte from the drug detection device; and
b) applying the drug detection device to the same or a different surface suspected of containing the target analyte.

20. A method of forming a drug detection device for detecting a target analyte on a surface, the method comprising the steps of:
a) forming a drug-detecting composition comprising:
i) one or more color-changing ingredients that produces variable visible color changes upon exposure to a target analyte;
ii) a binder component;
iii) at least one surfactant; and
iv) a solvent; and
b) applying the drug-detecting composition to a substrate.
